(19) United States
(12) Patent Application Publication
(10) Pub. No.: US 2018/0026470 A1
(43) Pub. Date: Jan. 25, 2018

(54) External Charger for an Implantable Medical Device Having a Conductive Layer Printed or Deposited on an Inside Housing Surface

(71) Applicant: Boston Scientific Neuromodulation Corporation, Valencia, CA (US)

(72) Inventor: Daniel Aghassian, Glendale, CA (US)

(21) Appl. No.: 15/643,063

(22) Filed: Jul. 6, 2017

Related U.S. Application Data
(60) Provisional application No. 62/365,098, filed on Jul. 21, 2016.

(51) Int. Cl. H02J 7/02; H02J 7/00; H02J 50/10; A61N 1/378

(52) U.S. Cl. CPC H02J 7/025; A61N 1/3787; H02J 7/0042; H02J 50/10

(57) ABSTRACT

A charging system for an Implantable Medical Device (IMD) is disclosed having a charging coil and one or more sense coils. The charging coil and one or more sense coils are preferably housed in a charging coil assembly coupled to an electronics module by a cable. The charging coil is preferably a wire winding, while the one or more sense coils are preferably formed in a conductive layer printed or deposited on an inside surface of the charging coil assembly housing or on an insulative substrate in contact with the inside surface. The conductive layer may also form traces in the charging coil assembly to couple to various electronic components within the housing, including for example a tuning capacitor for the charging coil, and one or more temperature sensors.

### CROSS REFERENCE TO RELATED APPLICATIONS

This is a non-provisional application of U.S. Patent Application Ser. No. 62/365,098, filed Jul. 21, 2016, to which priority is claimed, and which is incorporated by reference in its entirety.

### FIELD OF THE INVENTION

The present invention relates to wireless external chargers for use in implantable medical device systems.

### BACKGROUND

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system, including a Deep Brain Stimulation (DBS) system.

As shown in FIGS. 1A-1C, an SCS system typically includes an Implantable Pulse Generator (IPG) 10 (Implantable Medical Device (IMD) 10 more generally), which includes a biocompatible device case 12 formed of a conductive material such as titanium for example. The case 12 typically holds the circuitry and battery 14 (FIG. 1C) necessary for the IMD 10 to function, although IMDs can also be powered via external RF energy and without a battery. The IMD 10 is coupled to electrodes 16 via one or more electrode leads 18, such that the electrodes 16 form an electrode array 20. The electrodes 16 are carried on a flexible body 22, which also houses the individual signal wires 24 coupled to each electrode. In the illustrated embodiment, there are eight electrodes (Ex) on each lead 18, although the number of leads and electrodes is application specific and therefore can vary. The leads 18 couple to the IMD 10 using lead connectors 26, which are fixed in a non-conductive header material 28, which can comprise an epoxy for example.

As shown in the cross-section of FIG. 1C, the IMD 10 typically includes a printed circuit board (PCB) 30, along with various electronic components 32 mounted to the PCB 30, some of which are discussed subsequently. Two coils (more generally, antennas) are shown in the IMD 10: a telemetry coil 34 used to transmit/receive data to/from an external controller (not shown); and a charging coil 36 for charging or recharging the IMD's battery 14 using an external charger, discussed next.

FIG. 2A shows the IMD 10 in communication with an external charger 50 used to wirelessly convey power to the IMD 10, which power can be used to recharge the IMD's battery 14. The transfer of power from the external charger 50 is enabled by a primary charging coil 52. The external charger 50, like the IMD 10, also contains at least one PCB on which electronic components 56 are placed. Some of these electronic components 56 are discussed subsequently. In the example shown, the external charger 50 includes two PCBs: a horizontal PCB 54, and a vertical PCB 55. This can be beneficial because conductive electronics components 56 can be placed on the vertical PCB 55, and thus will be less susceptible interference from the magnetic field 66 produced by the charging coil 52. For example, placing electronics components 56 on the vertical PCB 55 reduces the production of Eddy currents in the components, which reduces energy loss and unnecessary heating, as explained in U.S. Pat. No. 9,002,445. Horizontal PCB 54 by contrast preferably includes only the charging coil 56 itself. This arrangement however is not strictly necessary; the external charger 50 may include only a single horizontal PCB 54 which also includes the electronic components 56, as the '445 patent explains.

A user interface 58, including touchable buttons and perhaps a display and a speaker, allows a patient or clinician to operate the external charger 50. A battery 60 provides power for the external charger 50, which battery 60 may itself be rechargeable. The external charger 50 can also receive AC power from a wall plug. A hand-holdable housing sized to fit a user's hand contains all of the components, and in the example of FIG. 2A the external charger 50's housing is constructed of a top housing portion 62a and a bottom housing portion 62b.

Power transmission from the external charger 50 to the IMD 10 occurs wirelessly and transcutaneously through a patient's tissue 25 via inductive coupling. FIG. 3 shows details of the circuitry used to implement such functionality. Primary charging coil 52 in the external charger 50 is energized via charging circuit 64 with an AC current, Icharge, to create an AC magnetic charging field 66. This magnetic field 66 induces a current in the secondary charging coil 36 within the IMD 10, providing a voltage across coil 36 that is rectified (38) to DC levels and used to recharge the battery 14, perhaps via a battery charging and protection circuitry 40 as shown. The frequency of the magnetic field 66 can be about 80 kHz for example. When charging the battery 14 in this manner, is it typical that the housing of the external charger 50 (specifically, bottom housing portion 62b) touches the patient's tissue 25, perhaps with a charger holding device or the patient's clothing intervening, although this is not strictly necessary.

Vcoil formed across the external charger's charging coil 52 in response to charging current Icharge can also be assessed by alignment circuitry 70 to determine how well the external charger 50 is aligned relative to the IMD 10. This is important, because if the external charger 50 is not well aligned to the IMD 10, the magnetic field 66 produced by the charging coil 52 will not efficiently be received by the charging coil 36 in the IMD 10. Efficiency in power transmission can be quantified as the “coupling” between the transmitting coil 52 and the receiving coil 36 (k, which ranges between 0 and 1), which generally speaking comprises the extent to which power expended at the transmitting coil 52 in the external charger 50 is received at the receiving coil 36 in the IMD 10. It is generally desired that the coupling between coils 52 and 36 be as high as possible: higher coupling results in faster charging of the IMD battery 14 with the least expenditure of power in the external charger 50. Poor coupling is disfavored, as this will require high power drain (e.g., a high Icharge) in the external charger 50 to adequately charge the IMD battery 14. The use of high power depletes the battery 60 in the external charger 50, and more importantly can cause the external charger 50 to heat up, and possibly burn or injure the patient.

Generally speaking, if the external charger 50 is well aligned with the IMD 10, then Vcoil will drop as the charging circuitry 64 provides the charging current Icharge to the charging coil 52. Accordingly, alignment circuitry 70 can compare Vcoil, preferably after it is rectified 76 to a DC voltage, to an alignment threshold, Vt. If Vcoil<Vt, then external charger 50 considers itself to be in good alignment with the underlying IMD 10. If Vcoil>Vt, then the external charger 50 will consider itself to be out of alignment, and can indicate that fact to the patient so that the patient can attempt to move the charger 50 into better alignment. For example, the user interface 58 of the charger 50 can include a position indicator 74. The position indicator 74 may comprise a speaker (not shown), which can “beep” at the patient when misalignment is detected. Position indicator 74 can also or alternatively include one or more Light Emitting Diodes (LED(s); not shown), which may similarly indicate charger-to-IMD position. Although not shown, Vcoil can be reduced in magnitude by a voltage divider (e.g., resistor ladder) before being presented to the alignment circuitry 70.

Vcoil may also be assessed to determine data telemetered from the IMD 10 to the external charger 50. In this regard, Vcoil (again possibly as reduced) may be presented to demodulation circuitry 68. In this example, telemetry from the IMD 10 may occur using Load Shift Keying (LSK), in which different logical bits (‘0’ and ‘1’) are formed at the IMD 10 by modulating the impedance of the receiving charging coil 36. Thus, LSK data to be transmitted can be sent to transistors 44 to selectively short or not short (‘1’ or ‘0’) the coil 36 to ground, or to a transistor 46 to selectively close or open the coil. This impedance modulation affects Vcoil at the external charger 50 due to the mutual inductance between the coils 52 and 36, with Vcoil being higher upon transmission of a ‘1’ bit, and lower upon transmission of a ‘0’ bit. Demodulation circuitry 68 can thus assess this difference in Vcoil magnitude to resolve whether ‘0’ or ‘1’ bits are presently being transmitted from the IMD 10 in a sequential bit stream.

External charger 50 can also include one or more thermistors 71, which can be used to report the temperature (expressed as voltage Vt in FIG. 3) of external charger 50 to its control circuitry 72, which can in turn control production of the magnetic field 66 such that the temperature remains within safe limits. See, e.g., U.S. Pat. No. 8,321,029, describing temperature control in an external charging device using readings provided by thermistors. Thermocouples could also be used in place of the one or more thermistors 71. In the example of FIG. 2A, only one thermistor 71 is shown for simplicity. Notice that the thermistor 71 is coupled to the horizontal PCB 54. Such positioning of the thermistor 71 is understandable, as many available thermistors are constructed to be mountable to a circuit board by well-known surface mounting techniques.

However, the inventor finds such positioning of the thermistor 71 in FIG. 2A to be problematic, because the thermistor 71 is not sensing the temperature at the point at which the external charger contacts the patient's tissue 25. Instead, the thermistor 71 senses the temperature at a distance d from the patient, which distance d largely comprises air that does not conduct heat well. As such, the temperature the thermistor 71 senses is not the temperature experienced by the patient during a charging session, which is the main concern from a safety perspective.

It is not necessary that thermistor(s) 71 be placed on a circuit board of the external charger 50. FIG. 2B shows an external charger 50′ similar in many respects to the external charger 50 of FIG. 2A, and similar element numerals are not reiterated. Notice however that one or more thermistors 71 in external charger 50′ have been placed on an inside surface of the bottom housing portion 62b (again, only one thermistor is shown). In this position, the thermistor 71 is better able to sense the temperature during a charging session at a location experienced by the patient, which is beneficial from a safety standpoint.

However, separating the thermistor 71 from the circuit board (e.g., PCB 54) is also problematic, because the external charger 50′ needs to include a connection between the thermistor 71 and the circuit board 54. In FIG. 2B, such connection comprises wires 73 connecting to the two terminals of the thermistor 71, which wires proceed through a central hole 57 in the PCB 54. Such wires 73 can be difficult to assemble, especially because they are most logically assembled by hand. Wires 73 can further be unreliable, as connections to either the thermistor(s) 71 or the PCB 54 can break. Further, once the external charger 50′ is assembled, it can be difficult to verify that the wires 73 are still properly connected to the thermistor.

Further, the external chargers 50 and 50′ determine position of the charger relative to the IMD 10 using measurements taken from the same charging coil 52 (e.g., Vcoil) used to produce the magnetic field. This too has drawbacks and limits the types of positioning measurements that can be made.

### BRIEF DESCRIPTION OF THE DRAWINGS

FIGS. 1A-1C show different views of an implantable pulse generator, a type of implantable medical device (IMD), in accordance with the prior art.

FIGS. 2A and 2B show external chargers being used to charge a battery in an IMD, while FIG. 3 shows circuitry in both, in accordance with the prior art.

FIGS. 4A and 4B show an improved charging system having a charging coil assembly and an electronics module connected by a cable, in which conductive layer traces are formed on the inside surface of the bottom housing portion of the charging coil assembly, in accordance with an example of the invention.

FIGS. 5A and 5B show traces formed in the conductive layer and how they can be mechanically and electrically connected to various electronic components and wires in the charging coil assembly, in accordance with an example of the invention.

FIG. 6 shows a circuit diagram including various circuitry in the charging coil assembly and the electronics module, in accordance with an example of the invention.

FIGS. 7A and 7B show use of a connector to connect wires in the cable to various traces in the conductive layer, in accordance with an example of the invention.

FIG. 8 shows an alternative in which the conductive layer is printed or deposited on an insulative substrate, which substrate is then placed on the inside surface of the bottom housing portion of the charging coil assembly, in accordance with an example of the invention.

FIG. 9 shows use of the disclosed conductive layer in an integrated external charger in which the electronics, charging coil, and sense coil(s) are housed in a single housing, in accordance with an example of the invention.

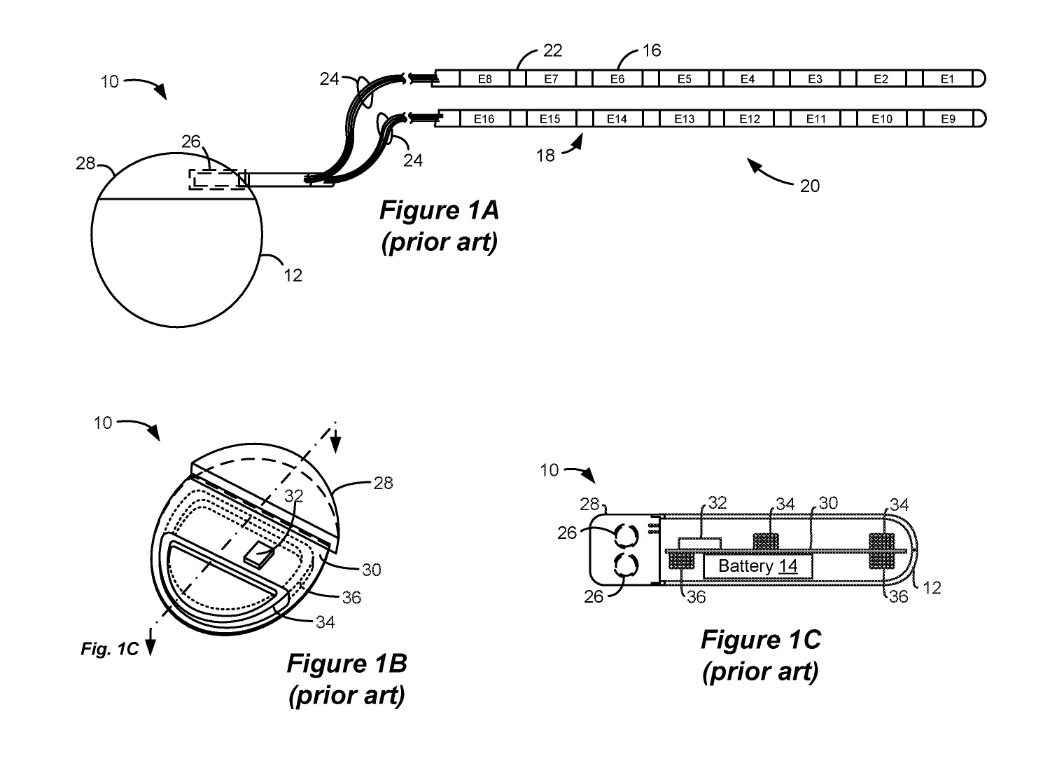

### DETAILED DESCRIPTION

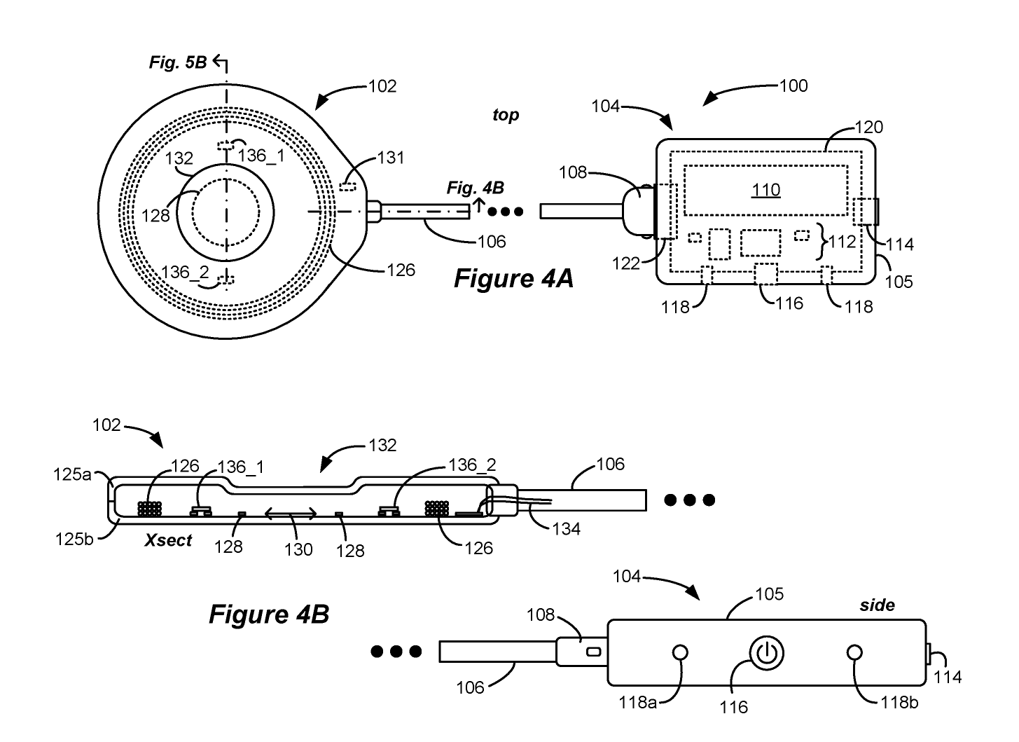

An improved charging system 100 for an IMD 10 is shown in FIGS. 4A and 4B. Charging system 100 includes two main parts: an electronics module 104 and a charging coil assembly 102 which includes a charging coil 126. The electronics module 104 and the charging coil assembly 102 are connected by a cable 106. The cable 106 may be separable from both the electronics module 104 and the charging coil assembly 102 via a port/connector arrangement, but as illustrated cable 106 is permanently affixed to the charging coil assembly 102. The other end of the cable 106 includes a connector 108 that can attach to and detach from a port 122 of the electronics module 104.

Electronics module 104 preferably includes within its housing 105 a battery 110 and active circuitry 112 needed for charging system operation, some of which are described subsequently. Electronics module 104 may further include a port 114 (e.g., a USB port) to allow its battery 110 to be recharged in conventional fashion, and/or to allow data to be read from or programmed into the electronics module, such as new operating software. Housing 105 may also carry a user interface, which as shown in the side view of FIG. 4B can include an on/off switch to begin/terminate generation of the magnetic field 66, and one or more LEDs 118a and 118b. In one example, LED 118a is used to indicate the power status of the electronics module 104. For example, LED 118a may be lit when its battery 110 is charged, and may blink to indicate that the battery 110 needs charging. More complicated user interfaces, such as those incorporating a speaker and a display, could also be used. User interface elements can be included on other faces of the electronic module's housing 105, and may be placed such that they are easily viewed for the therapeutic application at hand (e.g., SCS, DBS). Electronics are integrated within the housing 105 of the electronics module 104 by a circuit board 120.

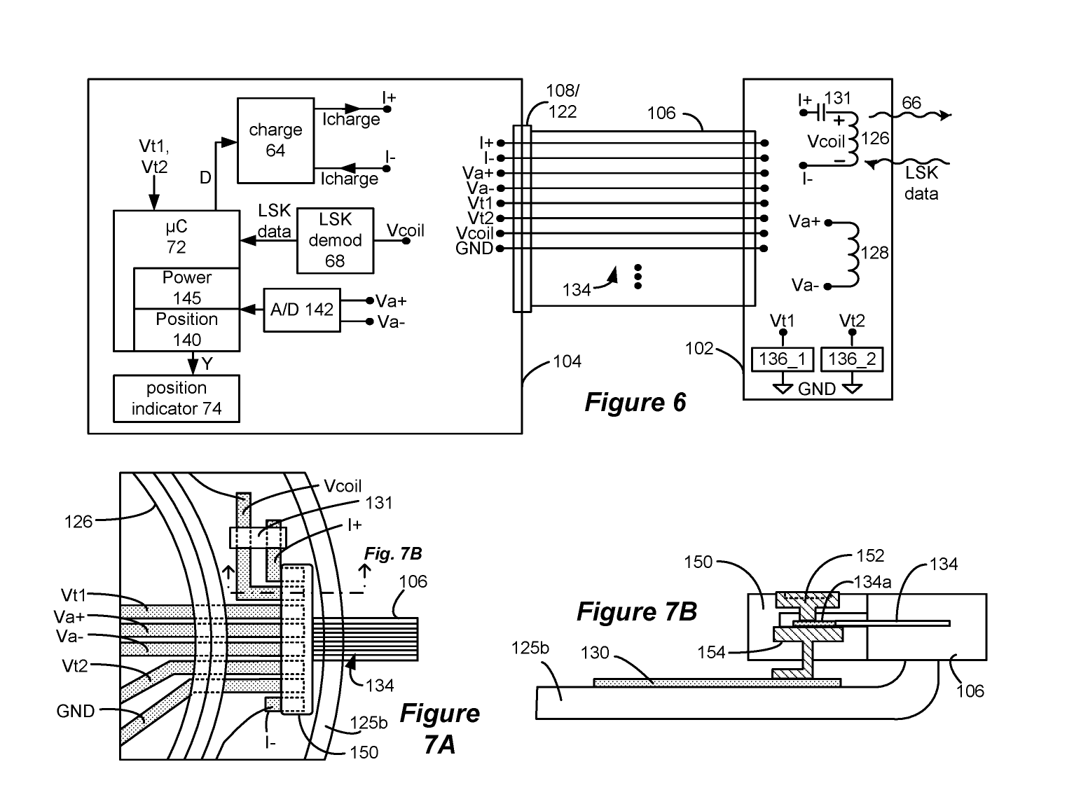

Charging coil assembly 102 preferably contains only passive electronic components that are stimulated or read by active circuitry 112 within the electronics module 104. Such components include the primary charging coil 126 already mentioned, which as illustrated comprises a winding of copper wire and is energized by charging circuitry 64 (FIG. 6) in the electronics module 104 to create the magnetic charging field 66 that provides power to the IMD 10, such as may be used to recharge the IMD 10's battery 14.

Components in the charging coil assembly 102 are integrated within a housing, which may be formed in different ways. In one example, the housing may include top and bottom housing portions 125a and 125b formed of hard plastic that can be screwed, snap fit, ultrasonic welded, or solvent bonded together. Alternatively, assembly housing may include one or more plastic materials that are molded over the electronics components. One side of the housing (e.g., the top portion 125a) may include an indentation 132 to accommodate the thickness of a material (not shown) that can be useful to affixing the charging coil assembly 102 to the patient, to the patient's clothes, or within a holding device such as a charging belt or harness. See, e.g., U.S. Patent Application Publication 2016/0301239, disclosing a belt for holding a charging coil assembly and control module that can be used with charging system 100. Such material may include Velcro or double-sided tape for example. Preferably, bottom housing portion 125b touches or faces the patient when the charging system 100 is used during a charging session to provide power to the IMD 10.

Further included within the charging coil assembly 102 are one or more sense coils 128, although only one is shown in FIGS. 4A and 4B. The one or more sense coils 128 are measured in various ways to perform different functions in the charging system 100. For example, sense coil measurements can be used to determine the position of the charging coil 126 (charging coil assembly 102) with respect to the IMD 10 being charged, and more specifically whether the charging coil 126 is aligned and/or centered with respect to an IMD 10 being charged, as disclosed in U.S. Provisional patent application Ser. No. 15/616,463, filed Jun. 7, 2017, which is incorporated herein by reference in its entirety. Sense coil measurements can also be used to adjust the power of the magnetic field 66 provided by the charging coil 126, and may further be used to adjust the frequency of the magnetic field 66 to a resonant frequency of the charger/IMD system, again as explained in the '463 Application. Notice that the one or more sense coils 128 may be made concentric with the charging coil 126, and may be formed with a smaller radius than the charging coil 126.

As shown in the cross section of FIG. 4B, the charging coil assembly 102 preferably does not contain a traditional printed circuit board. Instead, conductive connections necessary within the charging coil assembly 102, and the one or more sense coils 128, are formed in a conductive layer 130 in contact with and formed on the inside surface of the insulative bottom case portion 125b. The conductive layer 130 can be formed using several different techniques, including screen printing, flexography, gravure, offset lithography, inkjet lithography, and vapor deposition lithography. In short, the conductive layer 130 may comprise a printed or deposited layer on the inside surface. Depending on the technique used to form the conductive layer 130, the layer may comprise conductive inks, organic semiconductors, inorganic semiconductors, metallic conductors, nanoparticles, nanotubes, etc. Use of a conductive layer 130 is preferred over traditional PCB technologies for its low cost.

In the figures, the conductive layer 130 is shown as being placed only on flat portions of the inside surface of the bottom housing portion 125b. However, this isn't necessary. The inside surface of the bottom housing portion 125b need not be flat, and the conductive layer 130 can be formed on non-flat portions of the inside surface, including on the curved inside surfaces that form vertical walls at the periphery of the bottom housing portion 125b. Conductive layer 130 may also be formed in contact with the inside surface of the top housing portion 125a to connect to various electronic components, although this is not shown. Finally, conductive layer 130 could also be formed on outside surfaces of either of the housing portions 125a or 125b, although this isn't shown.

Components within the charging coil assembly 102 can be mechanically and electrically coupled to the conductive layer 130. For example, the charging coil assembly 102 preferably includes one or more tuning capacitors 131, shown also in the circuit diagram of FIG. 6. One capacitor 131 is shown, which is coupled to the charging coil 126 to tune the resonant frequency of this L-C circuit (e.g., to 80 kHz). One skilled in the art will understand that the value of the capacitor 131 (C) connected to the charging coil 126 can be chosen depending on the inductance (L) of that coil, in accordance with the equation f(res)=1/sqrt(2πLC). Each of the one or more sense coils 128 may also be coupled to a tuning capacitor 131, although this is not necessary and is not shown. A tuning capacitor 131 can be placed in series or in parallel with its associated coil, although a series configuration is shown in FIG. 6.

The charging coil assembly 102 can further include one or more temperature sensors 136, and two (136_1 and 136_2) are shown in the figures. Each is used to report the temperature of the charging coil assembly 102 (Vt1 and Vt2 respectively, FIG. 6) to the control circuitry 72 in the electronics module 104. Such temperature data can in turn be used by control circuitry 72 to control production of the magnetic field 66 such that the temperature remains within safe limits. See, e.g., U.S. Pat. No. 8,321,029, describing temperature control in an external charging device. Reported temperature data from the one of more temperature sensors may be averaged, and the temperature sensors can be placed in different positions within the charging coil assembly 102, including in the middle of the charging coil 126. Temperature sensors 136_1 and 136_2 may comprise thermistors, thermocouples, or other devices.

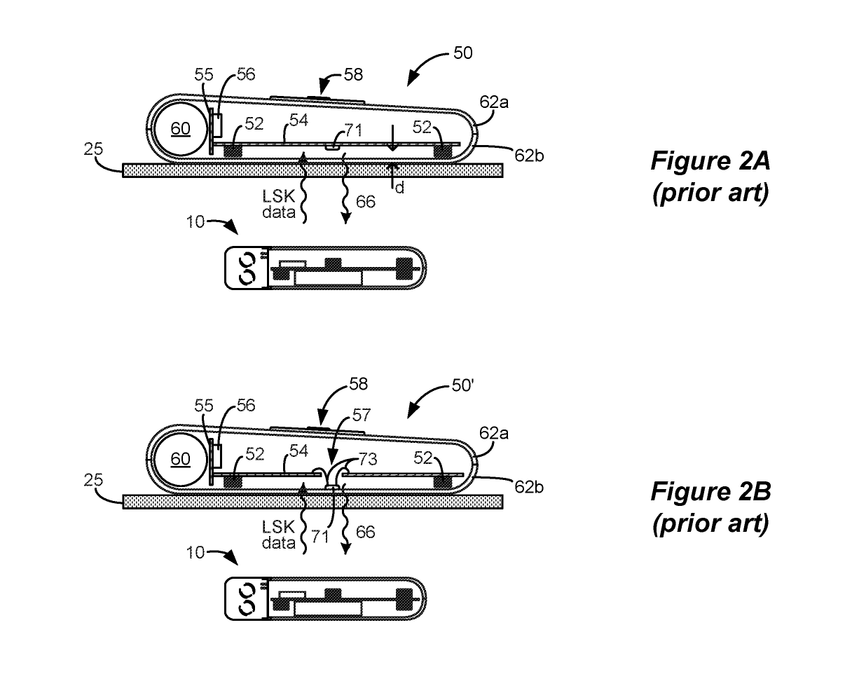

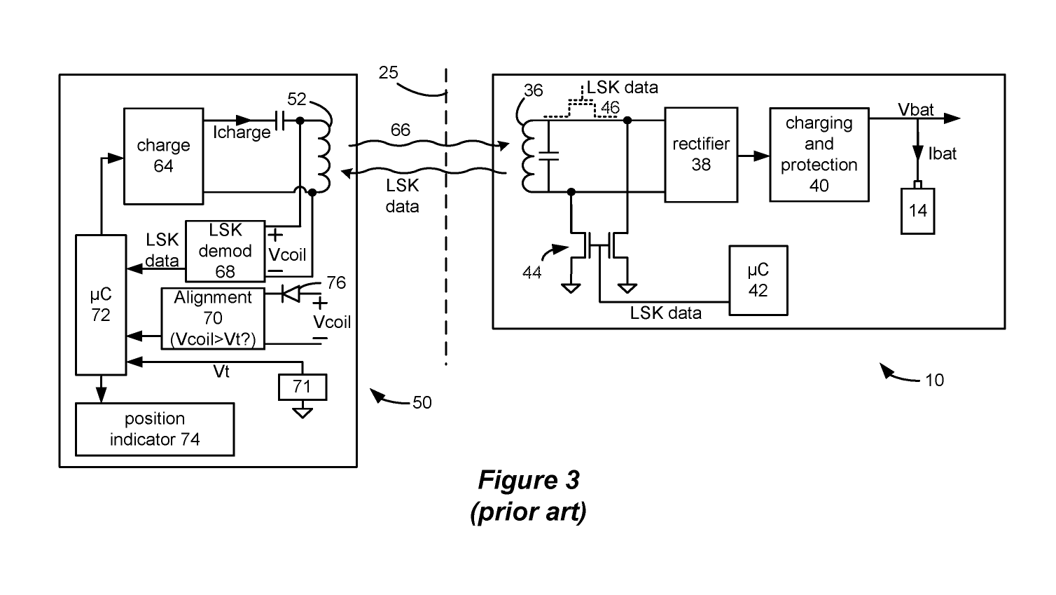

Notice in FIG. 4B that the temperature sensors 136_1 and 136_2 can be said to be in contact with the inside surface of the bottom housing portion 125b by virtue of their connections to the conductive layer 130—which layer 130 is itself in contact with the inside surface, is relatively thin, and will reasonably conduct heat by virtue of its conductivity. This is beneficial compared to external chargers 50 and 50′ discussed in the Background with respect to FIGS. 2A and 2B. Such positioning of temperature sensors 136_1 and 136_2 allows them to better sense the temperature during a charging session at a location experienced by the patient (inside the bottom housing portion 125b that touches or faces the patient), unlike external charger 50 of FIG. 2A but like external charger 50′ of FIG. 2B, which is beneficial from a safety standpoint. This benefit is however achieved without the use of wires—such as wires 73 of FIG. 2B—which are unreliable and difficult to assemble. If desired, thermally conductive epoxy or like materials can be used to further promote heat conduction to the temperature sensors 136_1 and 136_2 and to further affix them in contact with the inside surface.

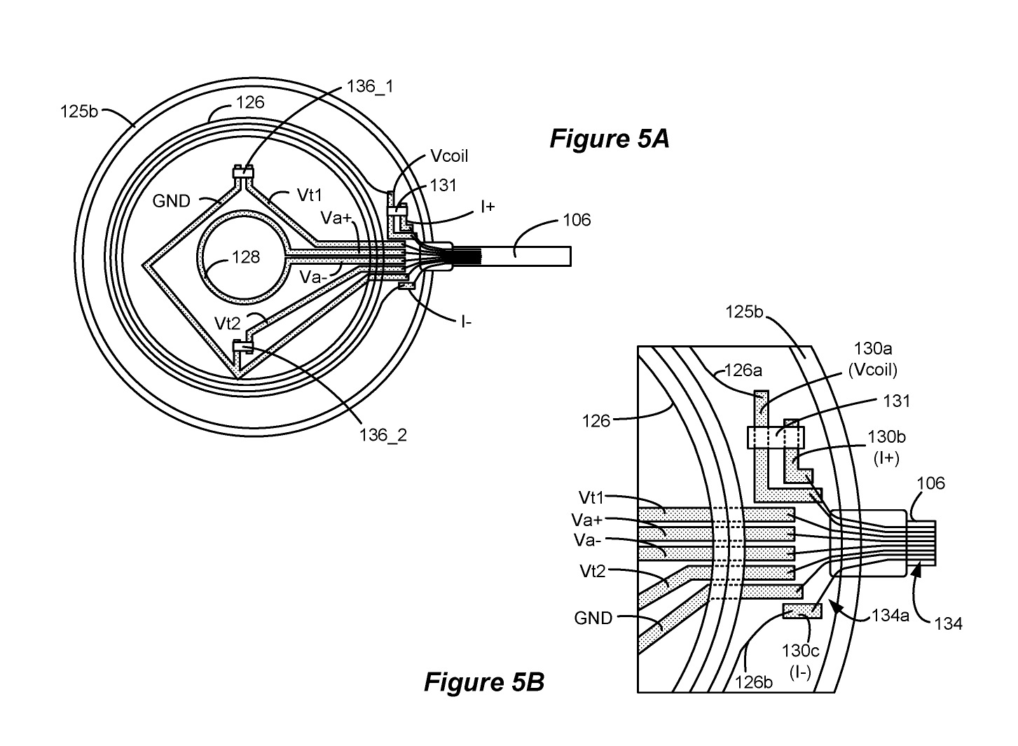

FIGS. 5A and 5B show the charging coil assembly 102 with the top housing portion 125a removed to reveal how conductive layer 130 can be formed into traces on the inside surface of the bottom case portion 125b, and connected to relevant signal wires 134 in the cable 106 and to the components (e.g., charging coil 126, capacitor 131, temperature sensors 136_1 and 136_2). The circuit diagram of FIG. 6 illustrates some of the signals that are passed between the electronics module 104 and the charging cable assembly along the signal wires 134. These signals include: I+ and I−, which comprise differential signals generated by the charging circuitry 64 in the electronics module 104 to drive the charging coil 126 with AC current Icharge; Va+ and Va−, which comprise a differential voltage that is induced across the sense coil 128 in the charging coil assembly 102; Vt1 and Vt2, which comprise the temperature voltages reported from the temperature sensors 136_1 and 136_2 in the charging coil assembly 102 to the control circuitry 72 in the electronics module 104; Vcoil, which comprises the voltage that builds across the charging coil 126 in response to current Icharge; and a ground signal (GND). I+/I− and Va+/Va− need not comprise differential signal, but could instead comprise single-ended signals referenced to ground. Similarly, Vcoil could comprise a differential signal rather than a single-ended signal.

Control circuitry 72 can comprise a microcontroller programmed with firmware, such as any of the STM32F4 ARM series of microcontrollers provided by STMicroeletronics, Inc., as described at http://www.st.com/content/st_com/en/products/microcontrollers/stm32-32-bit-arm-cortex-mcus/stm32f4-series.html ? querycriteria=productId=SS1577. Control circuitry 72 may also comprise an FPGA, DSP, or other similar digital logic devices, or can comprise analog circuitry at least in part as explained further below. Control circuitry 72 can further comprise a memory programmed with firmware and accessible to a microcontroller or other digital logic device should that logic device not contain suitable on-chip memory.

As explained in the above-incorporated '463 Application, Vcoil across the charging coil 126 can be monitored to determine data telemetered from the IMD 10, which data may transmitted by the IMD 10 using Load Shift Keying (LSK) telemetry. Vcoil may be reduced in magnitude by a voltage divider (e.g., resistor ladder) in the charging coil assembly 102 (not shown) before being transmitted along cable 106 to the electronics module 104. Vcoil is received at a LSK demodulator 68 to recover the transmitted data and to report it to the control circuitry 72, as described earlier.

The voltage induced across the sense coil 128 is affected by a position of the charging coil 126 with respect to the IMD 10, and is represented by differential AC signals Va+/Va− (or Va more simply, where Va=Va+−Va−). Va is received at one or more analog-to-digital (A/D) converters 142, with digital values being reported to either or both of a position module 140 and a power module 145. Modules 140 and 145 may comprise firmware programmed in the control circuitry 72.

As explained in detail in the above-incorporated '463 Application, one or more parameters determined from Va—including the maximum magnitude of Va, a phase angle between Va and a drive signal D used to energize the charging circuitry 64, or a resonant frequency of the charger/IMD system—can be used by position module 140 to determine the position of the charging coil 126 (or charging coil assembly 102 more generally) relative to the IMD 10, which position may include both a radial offset and a depth between the two. Such position information may be used by position module 140 to determine, for example, whether the charging coil 126 is centered (well coupled), misaligned (poorly coupled), or in an intermediate state (not centered but not misaligned) with respect to the IMD 10. Such position conditions may be indicated using position indicator 74, which may be similar to that described earlier. The same one or more parameters determined from Va may also be used by power module 145 to adjust the power of the magnetic field 66 that the charging coil 126 produces, or to adjust the frequency of the magnetic field 66 to resonance to render energy transfer to the IMD 10 maximally efficient. Such power adjustment may comprise varying a duty cycle of the drive signal D, while frequency adjustment may comprise varying a frequency of the drive signal D.

It should be noted that forming the least one sense coil 128 as a trace in the conductive layer 130 is effective, even if the conductivity and inductance of coils so formed are lower than a traditional wire winding. This may result in Va being relatively small (on the order of 0-3 Volts), but such signal strength is still sufficient to determine position and adjust charging coil power as just described. Va can be increased if necessary by increasing the area encompassed by the sense coil 128, or by including a greater number of turns. In this regard, the at least one sense coil 128 can comprise a multi-turn spiral as formed on the inside surface of the bottom housing portion 125b. If necessary, a jumper wire can be used to access the end of the sense coil 128 within the spiral.

Components such as the capacitor 131 and the temperature sensors 136_1 and 136_2 may comprise surface mountable components which may be mechanically and electrically connected to traces formed in the conductive layer 130 to establish the circuitry shown in FIG. 6. The manner in which such connections are made may depend on the materials used in the conductive layer 130. For example, connections between the components and the conductive layer 130 may be made by soldering, by the use of conductive epoxies, or by other known means. Notice in FIG. 5A that temperature sensor 136_1 is coupled between conductive layer 130 traces Vt1 and ground, and temperature sensor 136_2 is coupled between conductive layer 130 traces Vt2 and ground, as specified by the circuit diagram of FIG. 6. Capacitor 131 is coupled between conductive layer 130 traces for I+ and Vcoil, as explained further below. The charging coil 126 is preferably formed of an insulated wire winding, and hence the charging coil 126 may be affixed to the inner surface of the bottom housing portions 125b such that it overlays the conductive layer 130 traces without shorting to them.

The ends 126a and 126b of the charging coil 126 and the ends 134a of the wires 134 in cable 106 can be stripped and connected to appropriate traces in the conductive layer 130, as best seen in the magnified view of FIG. 5B. For example, and consistent with the circuit diagram of FIG. 6, it is seen that end 126a of the charging coil 126 is connected to a conductive layer trace 130a, which trace is also connected to the end of the wire 134 in cable 106 that carries signal Vcoil. Conductive layer trace 130a (Vcoil) is also coupled through capacitor 131 to a conductive layer trace 130b, which trace is also connected to the end of the wire 134 in cable 106 that carries signal I+. Conductive layer trace 130c is connected to both the end 126b of the charging coil 126 and the wire 134 in the cable 106 that carries I−.

It should be noted that other electronic components in the charging system 100 can be included within the charging coil assembly 102 and connected to the conductive layer 130. For example, control circuitry 72 and other circuitry 112 described as within the electronics module 104 can be coupled to conductive layer 130 traces in the charging coil assembly 102. Thus, electronics module 104 may retain only battery 110 and user interface aspects. Other electronic components mountable to or formable in the conductive layer 130 may include short-range radio-frequency (e.g., Bluetooth) antennas and/or related telemetry circuitry, resistors, etc. LEDs could also comprise an electronic component connected to the conductive layer 130, which LEDs could either shine through the bottom housing portion 125b (if made of a translucent material), or proceed through holes formed in the bottom housing portion 125b.

FIGS. 7A and 7B illustrate another manner in which connections can be made between the wires 134 in cable 106 and the various conductive layer 130 traces on the inside surface of the bottom housing portion 125b. In these figures, the stripped ends 134a of wires 134 terminate at a connector 150 which is mechanically and electrically connected to the conductive layer 130 traces. As shown best in FIG. 7B, stripped ends 134a of the wires 134 are held connected to conductive contacts 154 within the connector 150. In the example shown, the stripped ends 134a are held by screws 152 which may be turned to press the ends 134a against the contacts 154. However, other means of connection may be used, such as latches, biased springs, etc. The contacts 154 extend through the bottom of the connector 150, where they can be mechanically and electrically connected to the conductive layer 130 traces. Again, connection can be made by solders, conductive epoxies and the like.

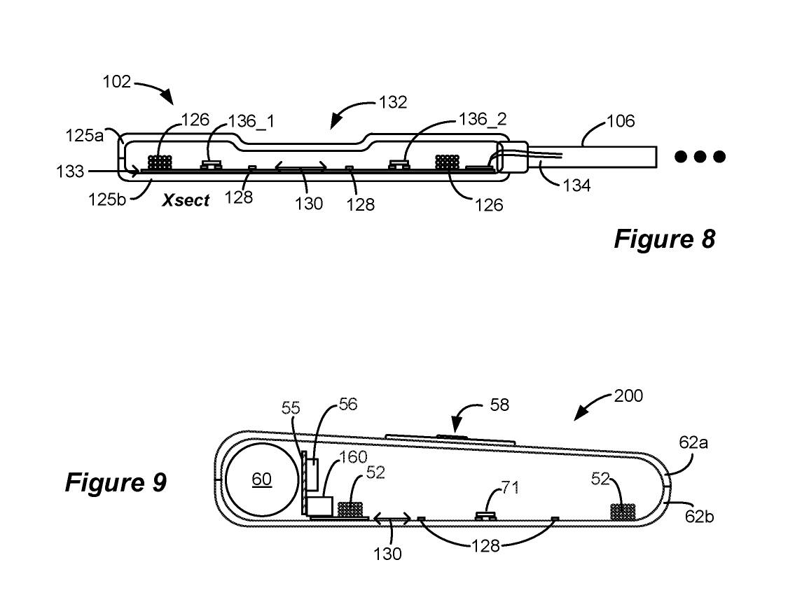

It is not strictly necessary that the conductive layer 130 be directly in contact with and formed on the inside surface of the bottom housing portion 125b. Instead, the conductive layer 130 may be printed or deposited on a conductive layer substrate 133 as shown in FIG. 8. Conductive layer substrate 133 may comprise an insulative material upon which the conductive layer 130 may be printed or deposited. For example, conductive layer substrate 133 could comprise a Kapton™ film, or other thin insulative substrates. Additionally, conductive layer substrate 133 may be flexible or rigid. In any event, the conductive layer 130 (for example, the at least one sense coil 128 and the various conductive layer traces) can be formed on the conductive layer substrate 133, and then directly affixed to the inside surface of the bottom housing portion 125b (e.g., using an adhesive). This alternative may be preferred for example when it would be easier to form the conductive layer 130 structures on a flat surface rather than directly onto the bottom housing surface itself.

While the disclosed techniques employing printed or deposited traces and/or sense coils are described in the context of a charger system 100 having a separate electronics module 104 and charging coil assembly 102 (see FIGS. 4A and 4B), this is not necessary. Instead, the described techniques can also be implemented in an integrated external charger in which electronics, charging coil, and one or more sense coils are housed together. For example, FIG. 9 shows such an integrated external charger 200 with all components housed in a single housing having top and bottom housing portions 62a and 62b, which is generally similar to that described earlier in FIGS. 2A and 2B. Charger 50′ includes a conductive layer 130 on the inside surface of the bottom portion 62b, which both forms the sense coil 128 and traces to couple to other components such as thermistor 71 or like temperature sensor. Thus, it is not important to the disclosed technique that the charging/sense coils be separate from the electronics, or that they be housed in separate housings. Notice in FIG. 9 that a vertical PCB 55 may still be retained to which electronics components 56 may be mounted. Vertical PCB 55 can in turn be electrically and mechanically connected to conductive layer 130 traces using a connector 160. However, this is not strictly necessary: Charger 50′ may comprise no PCB, with electronic components 56 and battery 60 being electrically and mechanically connected to appropriate circuit nodes formed in traces in the conductive layer 130. Further, although not illustrated, external charger 50′ of FIG. 9 may further include use of the conductive layer substrate 133 of FIG. 8.

Although the charging coil 126 that produces the magnetic field 66 has to this point been disclosed as comprised of a wire winding, this is not strictly necessary. Instead, the charging coil 126 may also be formed in using the conductive layer 130 in contact with the inside surface of the bottom housing portion 125b, or on a conductive layer substrate like substrate like 133 depicted in FIG. 8. As with the sense coil 128, charging coil 126 when formed in the conductive layer 130 may comprise multiple turns.

While the disclosed techniques are described in the context of a charger system that is used to charge a battery 14 in an IMD 10, this is not strictly necessary. The disclosed charger systems can also be used to provide continuous magnetic fields 66 to power IMDs that lack batteries.

Referring to “a” structure in the attached claims should be construed as covering one or more of the structure, not just a single structure.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. An external charger for wirelessly providing energy to an implantable medical device (IMD), comprising:
a housing comprising at least one inside surface;
a charging coil within the housing, wherein the charging coil is configured to produce a magnetic field to wirelessly provide energy to the IMD; and
a conductive layer forming at least a sense coil, wherein the sense coil is configured to be induced by the magnetic field with an induced signal,
wherein the conductive layer is printed or deposited to be in contact with the at least one inside surface of the housing.

2. The external charger of claim 1, wherein the induced signal is affected by a position of the charging coil with respect to the 1 MB.

3. The external charger of claim 1, wherein the at least one sense coil is concentric with the charging coil.

4. The external charger of claim 3, wherein a radius of the at least one sense coil is smaller than a radius of the charging coil.

5. The external charger of claim 1, further comprising control circuitry configured to determine from one or more parameters determined from the induced signal a position of the charging coil with respect to the IMD.

6. The external charger of claim 5, further comprising a user interface configured to indicate to a user the determined position of the charging coil with respect to the 1 MB.

7. The external charger of claim 1, further comprising control circuitry configured to determine from one or more parameters determined from the induced signal a power adjustment or a frequency adjustment for the magnetic field.

8. The external charger of claim 1, wherein the conductive layer further forms a plurality of traces in contact with the at least one inside surface of the housing, and further comprising a temperature sensor connected to at least one of the one or more traces.

9. The external charger of claim 8, further comprising a capacitor electrically connected to the charging coil, wherein the capacitor is further connected to at least one of the plurality of traces.

10. The external charger of claim 8, further comprising an electronics module and a cable comprising a plurality of wires, wherein the housing is connected to the electronics module by the cable.

11. The external charger of claim 10, wherein an end of at least one of the wires is connected to at least one of the plurality of traces.

12. The external charger of claim 10, further comprising a connector, wherein an end of at least one of the wires is connected to the connector, and the connector is connected to at least one of the plurality of traces.

13. The external charger of claim 1, wherein the charging coil comprises a wire winding.

14. The external charger of claim 1, wherein the at least one inside surface comprises a bottom inside surface of a portion of the housing configured to touch or face a user during production of the magnetic field.

15. The external charger of claim 14, wherein the charging coil is affixed to the bottom inside surface.

16. An external charger for wirelessly providing energy to an implantable medical device (IMD), comprising:
a housing comprising at least one inside surface;
a charging coil within the housing, wherein the charging coil is configured to produce a magnetic field to wirelessly provide energy to the IMD;
a conductive layer comprising a plurality of traces, wherein the conductive layer is printed or deposited to be in contact with the at least one inside surface of the housing; and
at least one electronic component within the housing and connected to the plurality of traces.

17. The external charger of claim 16, wherein the at least one electronic component comprises a surface mountable capacitor electrically connected to the charging coil.

18. The external charger of claim 16, wherein the at least one electronic component comprises at least one temperature sensor, and further comprising control circuitry, wherein the at least one temperature sensor is configured to report at least one temperature measurement to the control circuit, and wherein the control circuitry is configured to control production of the magnetic field based on the at least one temperature measurement.

19. The external charger of claim 16, further comprising an electronics module and a cable comprising a plurality of wires, wherein the housing is connected to the electronics module by the cable, wherein an end of at least one of the wires is connected to at least one of the plurality of traces.

20. The external charger of claim 16, wherein the charging coil comprises a wire winding.

21. The external charger of claim 16, wherein the at least one inside surface comprises a bottom inside surface of a portion of the housing configured to touch or face a user during production of the magnetic field, wherein the charging coil is affixed to the bottom inside surface.

22. The external charger of claim 16, further comprising a thermally-conductive material to affix the at least one electronic component to the at least one inside surface.
